(19) United States
(12) Patent Application Publication
(10) Pub. No.: US 2005/0054931 A1
(43) Pub. Date: Mar. 10, 2005

(54) Tracking clutter filter for spectral & audio doppler

(76) Inventor: David W. Clark, Windham, NH (US)

Correspondence Address: PHILIPS INTELLECTUAL PROPERTY & STANDARDS, P.O. BOX 3001, BRIARCLIFF MANOR, NY, 10510, US

(21) Appl. No.: 10/933,551

(22) Filed: Sep. 3, 2004

Related U.S. Application Data
(60) Provisional application No. 60/501,529, filed on Sep. 9, 2003.

(51) Int. Cl. A61B 8/14

(52) U.S. Cl. 600/453; 600/437

(57) ABSTRACT

In an adaptive clutter filter for spectral Doppler imaging using an ultrasound system, the stopband center frequency and/or bandwidth of the clutter filter are effectively adjusted on a short time scale to better eliminate moving clutter while allowing low velocity bloodflow signals to pass through.

### CROSS REFERENCE TO RELATED CASES

Applicant claims the benefit of Provisional U.S. Application Ser. No. 60/501,529, filed Sep. 09, 2003.

### FIELD OF THE INVENTION

This invention relates to ultrasonic imaging systems and, in particular, to the elimination of clutter from echo signals received by an ultrasonic system in spectral Doppler imaging mode.

### DESCRIPTION OF THE RELATED ART

Ultrasonic medical transducers are used to observe the internal organs of a patient. The ultrasonic range is described essentially by its lower limit: 20 kHz, roughly the highest frequency a human can hear. The medical transducers emit ultrasonic pulses which, if not absorbed, echo (i.e., reflect), refract, or are scattered by structures in the body. Most of the received signal is from scattering, which is caused by many small inhomogeneities (much smaller than a wavelength) making a small part of the wave energy disperse in all directions. The signals are received by the transducer and these received signals are translated into images. The sum of the many scattered waves of random phase cause the resulting image of the received signals to be speckly.

There are a number of imaging and/or diagnostic modes in which an ultrasonic system operates. The most fundamental modes are A Mode, B Mode, M Mode, and 2D Mode. The A Mode is amplitude mode, where signals are displayed as spikes that are dependent on the amplitude of the returning sound energy. The B Mode is brightness mode, where the signals are displayed as various points whose brightness depends on the amplitude of the returning sound energy. The M Mode is motion mode, where B Mode is applied and a strip chart recorder allows visualization of the structures as a function of depth and time.

The 2D Mode is the fundamental two-dimensional imaging mode. In 2D mode, an ultrasonic transmission beam is swept back and forth so that internal structures can be seen as a function of depth and width. By rapidly steering the beams from left to right, 1 2D cross-sectional image may be formed. There are other imaging modes, which also image in two dimensions (and also in three dimensions), and these are often referred to by their own names, usually based on the type of technology/methodology (such as “harmonic” or “Doppler”) used to produce the image.

Several modes of imaging are dependent on the Doppler effect, the phenomena whereby the frequency of sound from an approaching object has a higher frequency and, conversely, sound from a receding object has a lower frequency. In ultrasonic systems, this effect is used to determine the velocity and direction of blood flow in a subject. Continuous wave (CW) Doppler mode transmits a continuous ultrasound signal and determines the frequency shift of the scattering echo received from moving targets, e.g., blood cells. By contrast, pulsed Doppler mode transmits a periodic pulse of ultrasound energy and determines the phase or time shift of the received series of pulse echoes, not on the frequency shift of a single echo. Major Doppler imaging techniques include color flow Doppler, spectral Doppler, and power Doppler.

In color flow imaging (CFI), sample volumes are detected and displayed utilizing color mapping for direction and velocity flow data. Most commonly, this results in a grey-scale image with superimposed colors indicating blood-flow velocity and direction. Color mapping formats include BART (Blue Away, Red Towards), RABT (Red Away, Blue Towards), or enhanced/variance flow maps where color saturations indicate turbulence/acceleration and color intensities indicate higher velocities. Some maps use a third color, green, to indicate accelerating velocities and turbulence. Aliasing (when the velocity of the blood flow being measured exceeds the Nyquist Limit (half the PRF)) can be used to detect flow disturbances, e.g., transitions from laminar to turbulent flow.

Power Doppler does not show the direction of flow, but rather the colors in a power Doppler image indicate whether any flow is present. The Doppler signals are processed differently in power Doppler imaging: instead of estimating mean frequency and variance through autocorrelation, the integral of the power spectrum is estimated and color-coded. Because power Doppler imaging is based on the total power of the received Doppler signal, the results are independent from the velocity of the blood-flow.

Spectral Doppler refers to ultrasound methods, whether pulsed or CW Doppler, which present the results of flow velocity measurements as a “spectral display”. A spectral display shows the entire Doppler frequency shift (or blood-flow velocity) range present in the measurements. Spectral Doppler usually also includes stereo audio output of the flow signal. An “amplitude vs. frequency spectral display” shows the amplitudes of all the Doppler frequency shifts present at a particular moment in time. The more common “time-velocity spectral display” shows how the full spectrum of Doppler frequency shifts (or blood-flow velocities) varies over time. FIG. 1 shows a time-velocity spectral display of a carotid artery. As can be seen in FIG. 1, the abscissa of the time-velocity spectral display represents time while the height represents speed (in cm/s).

In Doppler imaging modes, a high-pass filter must be used to reduce or eliminate high-amplitude, low-velocity signals from the in-coming signals. Because these unwanted strong and slow signals mostly come from the tissue walls (e.g., the heart, the liver, the walls of an artery or vein containing a blood flow), these high-pass filters are sometimes known as “wall filters”. Without high-pass filtering, high-amplitude, low velocity Doppler signals generally overwhelm low-amplitude, high-velocity signals, such as the weak and fast signals of a blood flow. Specifically, the unwanted strong and slow signals create clutter signals (high-amplitude spikes in the time-velocity spectrum) and “wall thump” in the audio speakers. These high-pass filters are also known as “clutter filters”.

When the high-pass filter is fixed, e.g., with its stopband centered at DC (i.e., zero frequency), moving clutter signals can still get past and disturb the flow measurements. Some color flow imaging ultrasound systems use “adaptive” clutter filters to eliminate these moving clutter signals as well. An adaptive clutter filter adapts (i.e., changes itself in real-time) based on the incoming signal.

In CFI, it is easy to implement a clutter filter as an adaptive filter. For one thing, the input signal is split up into “flow packets” in CFI, and it is easier to implement an adaptive filter for eliminating clutter on packets of data. For another thing, CFI only displays mean parameters of each flow packet (i.e., the end results of the signal processing do not need the individual samples which went in as input).

By contrast, implementing an adaptive clutter filter in spectral Doppler is more challenging. In CFI, the data packets are distinct and can be clutter filtered independently. In spectral Doppler, the packets, i.e., the fast Fourier Transform (FFT) time segments, that are used in spectral analysis are neither distinct nor independent. Furthermore, the time response of the clutter filters in spectral Doppler typically extends over multiple FFT time segments.

Moving clutter signals are annoying when performing spectral Doppler imaging. For example, when imaging a carotid artery, the strong systolic pulse tends to put a bright blob near the baseline of the spectral display and, if one is listening to the audio signal, a thump in the audio. Because an adaptive clutter filter is not available for ultrasound systems in spectral Doppler imaging mode, the operator typically manually increases the cutoff frequency of the clutter filter (i.e., widens the stopband) when moving clutter begins to show up as bright (high amplitude), low (low frequency) signals in the time-velocity spectral display. However, if the clutter filter stopband is coarsely manipulated by operator manual control in order to eliminate the systolic clutter thump, then the slow diastolic blood flow is more difficult to see and measure. Furthermore, the radial and/or lateral motions in the carotid artery change over the cardiac cycle, resulting in a clutter signal which is continually changing frequency and bandwidth over time. It is not possible to keep up with such changes manually.

U.S. Pat. No. 6,296,612 to Mo et al. (hereinafter referred to as the “Mo system” or “Mo filter”) describes an adaptive clutter filter for use in spectral Doppler imaging and is hereby incorporated by reference in its entirety. As shown in FIG. 2 (which is a reproduction of FIG. 3 of the Mo patent), the incoming signal in the Mo system is filtered by wall filter 10 before going to a spectrum analyzer which takes the Fast Fourier Transform (FFT) of the high-pass filtered signal. In addition, on another path the incoming signal is low-pass filtered by LPF 26 (in order to isolate the clutter signal), and then the total power of the low-pass filtered signal is computed at 28. If there is significant clutter present in the filtered signal, the mean and the variance of the clutter frequency are calculated at 34. Filter selection logic 36 selects the most suitable filter coefficients from the filter coefficient lookup table (LUT) 22 based on the estimated mean and variance of the clutter frequency.

However, the Mo system's constant changing of the IIR filter coefficients while also filtering the incoming signal can result in objectionable artifacts due to the filter state being inconsistent with the new filter coefficients and past input data. Re-initializing the IIR filter state whenever the filter coefficients change in the Mo system is not practical, because the re-initialization itself causes a transient in the output, and this transient cannot be placed at the boundary between FFT time segments because the segments overlap.

Therefore, there is a need for an adaptive clutter filter for spectral Doppler imaging which can be adaptable in real-time without creating objectionable artifacts.

### SUMMARY OF TH INVENTION

The present invention provides a method and system for adaptively filtering the clutter from an incoming signal in an ultrasound system in spectral Doppler imaging mode. In the inventive system and method, the stopband of the clutter filter is automatically adjusted on a short time scale (preferably at least 4 times a second) to better target the moving clutter signal for elimination while allowing low velocity blood echoes to pass through to the spectrum analyzer.

In the adaptive clutter filter according to the present invention, there are two components: the estimation of the clutter frequency and the filtering of the incoming signal. During estimation, instantaneous correlation estimates are formed and then averaged over a short period to produce average short-term correlation estimates. During filtering, the current average correlation estimate(s) is used to modify the input and/or output of the IIR clutter filter(s).

Other objects and features of the present invention will become apparent from the following detailed description considered in conjunction with the accompanying drawings. It is to be understood, however, that the drawings are designed solely for purposes of illustration and not as a definition of the limits of the invention, for which reference should be made to the appended claims. It should be further understood that the drawings are not necessarily drawn to scale and that, unless otherwise indicated, they are merely intended to conceptually illustrate the structures and procedures described herein.

### BRIEF DESCRIPTION OF THE DRAWINGS

In the drawings:

FIG. 1 is a time-velocity spectral display showing how the full spectrum of Doppler frequency shifts (or blood-flow velocities) varies over time in a carotid artery according to conventional spectral Doppler imaging;

FIG. 2 is a flowchart/block diagram showing the components/steps for a prior art adaptive IIR clutter filter in a spectral Doppler imaging system;

FIG. 3 is a flowchart/block diagram showing the components/steps for an adaptive clutter filter in a spectral Doppler imaging system according to the present invention;

FIG. 4 is a flowchart/block diagram showing the components/steps of the estimation module 100 from FIG. 3 according to a preferred embodiment of the present invention;

FIG. 5 is a flowchart/block diagram showing the components/steps of the filtering module 200 from FIG. 3 implemented as a center frequency adapting clutter filter according to a preferred embodiment of the present invention; and

FIG. 6 is a flowchart/block diagram showing the components/steps of the filtering module 200 from FIG. 3 implemented as a bandwidth adapting clutter filter according to a preferred embodiment of the present invention.

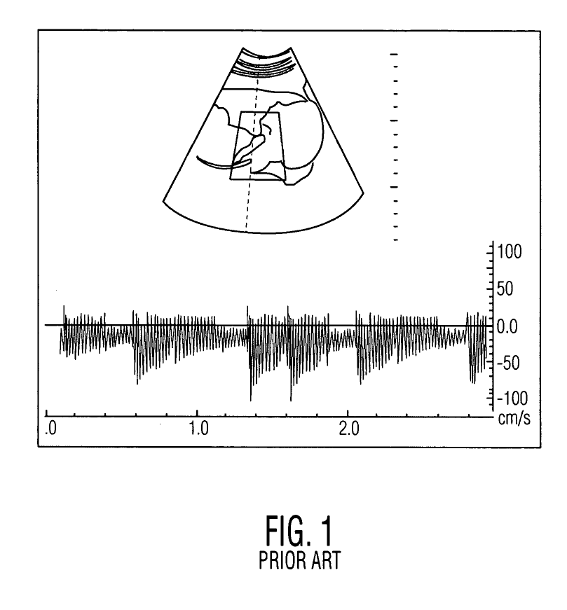

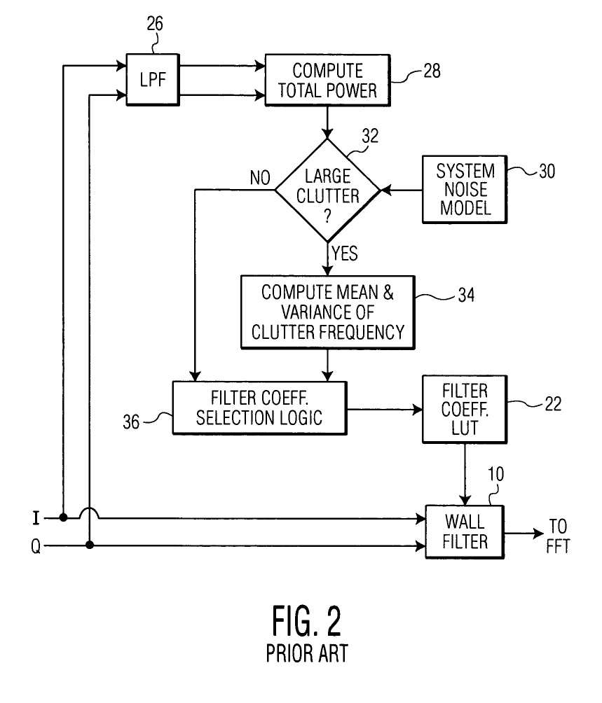

### DETAILED DESCRIPTION OF THIE PRESENTLY PREFERRED EMBODIMENTS

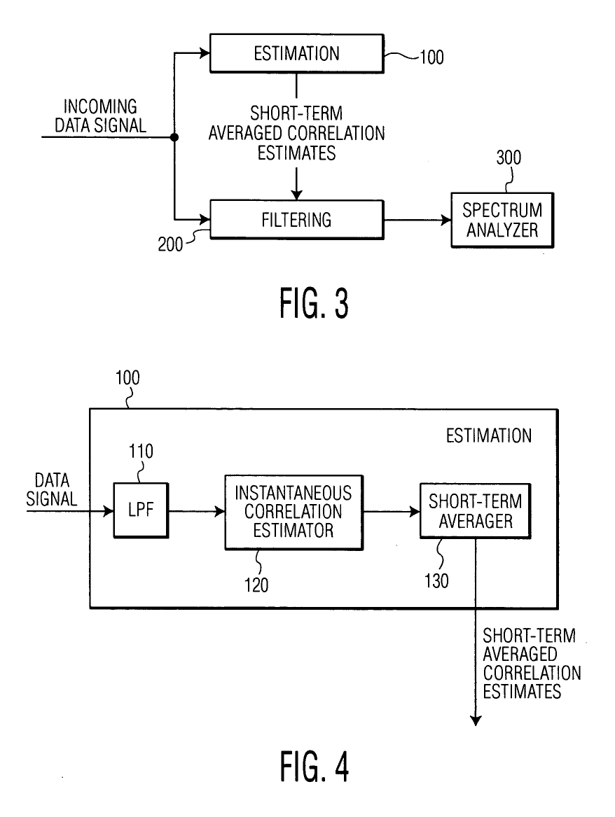

As stated above, the present invention is directed to an adaptive clutter filter with two basic components, as shown in FIG. 3: the estimation 100 of the clutter frequency and the filtering 200 of the incoming signal before entering the spectrum analyzer 300. It should be understood that these three modules are conceptual, and do not limit the manner of implementing the present invention in any way, i.e., the functions shown herein being performed in these modules may be performed by any combination of hardware, software, or firmware. Furthermore, the functions in one module may be performed by another, or combined together in a single module.

During estimation 100, instantaneous correlation estimates are formed and then averaged over a short period to produce average short-term correlation estimates. The specific components of Estimation 100 are shown in FIG. 4. Estimation 100 may include a low-pass filter (LPF) 110 which will filter the time-domain data signal so that only low frequency signals, where most of the power of the clutter signal is, are used to generate the clutter signal estimate. Next, an instantaneous estimator 120 forms instantaneous correlation estimates from the filtered signal. In the preferred embodiment of the present invention, the instantaneous estimator 120 forms instantaneous lag 1 correlation estimates by multiplying each sample by the conjugate of the previous sample. In other preferred embodiments, a lag greater than one sample may be used to provide better frequency resolution, particularly if the incoming clutter signal has first been low-pass filtered.

The instantaneous correlation estimates generated by the instantaneous estimator 120 are short-termed averaged by the short-term averager 130 in order to produce short-term averaged correlation estimates. The short-term averager can be implemented using, for example, either a moving average filter (FIR filter) or autoregressive (IIR filter) technique. The short-term averaged correlation estimates may be computed less often than every sample, provided that there is enough overlap of the averaging to ensure that successive estimates change gradually. The correlation estimates may be limited to low frequency (small angle) to avoid adapting to rapid motion, which would result in the adaptive clutter filter filtering out the desired signal, in unusual situations

Estimation 100 outputs short-term averaged correlation estimates. These correlation estimates are input to filtering 200, which uses them to adapt the one or more clutter filters on a short-time scale (preferably at least 4 times a second). Specifically, filtering 200 automatically adjusts the stopband center frequency of the clutter filter(s) and/or the width of the stopband itself in order to adapt the clutter filter(s) to the current clutter signal environment (as indicated by the correlation estimates from estimation 100). Thus, the two techniques of filter adaptation are (1) changing the location of the stopband center frequency on the spectrum and (2) increasing or decreasing the width of the stopband. Although these two adaptation techniques are presented separately here, a combination of both adaptation types may be used when implementing an adaptive clutter filter according to the present invention.

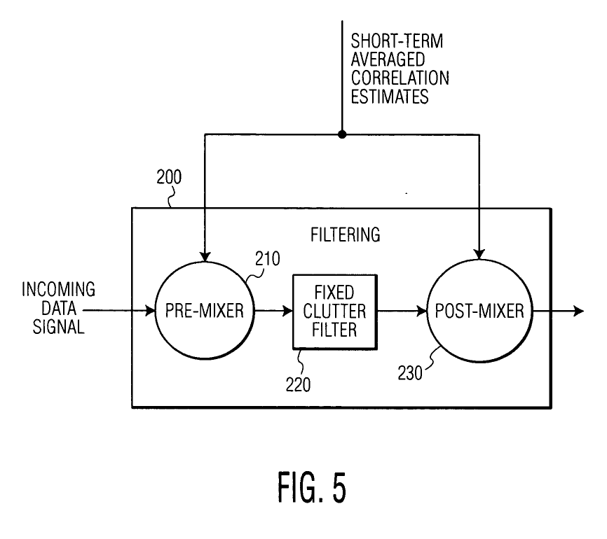

FIG. 5 shows filtering 200 being implemented as a center frequency adapting clutter filter. In FIG. 5, the incoming data signal is complex rotated (mixed) by pre-mixer 210 based on the correlation estimates from estimation 100. Essentially, the complex rotation causes the spectrum of the incoming signal to be moved either up or down in frequency. The shifted signal enters a ER clutter filter 220 which has real coefficients and has its stopband center frequency permanently set at DC (zero frequency). In other words, IIR clutter filer 220 is fixed in both bandwidth and center frequency. Essentially, the correlation estimates are used by mixer 210 to shift the incoming signal so that the clutter signal within the incoming signal will be centered at the center frequency of the fixed IIR clutter filter 220. In effect, this moves the stopband center frequency of IIR clutter filter 220 to where the clutter signal is, even though the IIR clutter filter 220 is not actually changed or adapted. The signal is moved, not the filter.

After IIR clutter filter 220 filters out the estimated clutter signal, the filtered signal is complex rotated (mixed) by post-mixer 230 back to its original frequencies based on the correlation estimates from estimation 100. In this embodiment, the complex rotation factor of post-mixer 230 is a variable-frequency local oscillator (LO), which is a unit-magnitude phasor whose phase is updated every sample by multiplying itself by the unit-magnitude version of the current correlation estimate. Consequently, the complex rotation factor of pre-mixer 210 is just the complex conjugate of the complex rotation factor of post-mixer 230, so that they have equal and opposite frequencies.

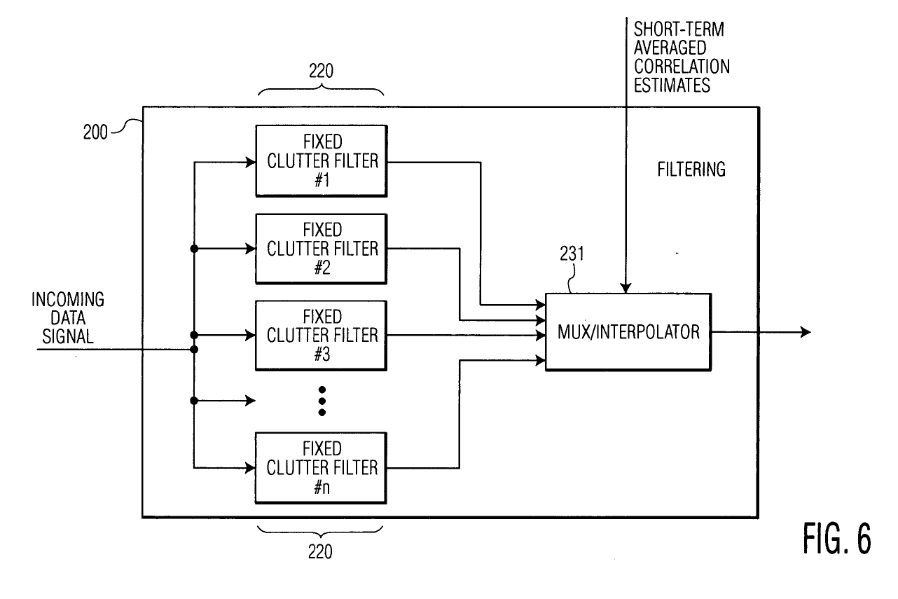

FIG. 6 shows an exemplary implementation of filtering 200 as a bandwidth adapting clutter filter. In FIG. 6, there is a bank of two or more IIR clutter filters 220, where each of the IIR clutter filters 220 has a stopband with a fixed frequency and width. Although the incoming data signal enters each of the IIR clutter filters 220, the outputs of the bank of clutter filters 220 enters MUX/Interpolator 231 which produces the filtered output signal. MUX/Interpolator 231 either selects the output from the most appropriate clutter filter from the bank of clutter filters 220, or generates an output signal by interpolating (blending) the outputs of two or more appropriate clutter filters. MUX/Interpolator 231 determines which clutter filters are appropriate based on the correlation estimates from estimation 100.

Both the data rotation technique for adapting frequency in FIG. 5 and the parallel filter technique for adapting bandwidth in FIG. 6 have IIR clutter filters with fixed stopbands. Thus, filtering 200 according to the preferred embodiments of the present invention avoids continually changing the IIR filter coefficients while filtering an ongoing incoming signal. In the prior art, dynamically changing the IIR filter coefficients while processing an ongoing incoming signal created objectionable artifacts due to the old clutter filter state being inconsistent with the new coefficients and past input data.

There are other adaptive IIR filter techniques which may change coefficients but avoid artifacts in other ways. For example, the new filter state may be estimated by filtering a finite set of input data, either past input data kept in a circular buffer, or forward input data from the current sample, if that is available. Or the new filter state may be analytically calculated from the old and new coefficients and the old state.

Any of the techniques described here may be combined, for example, to form a clutter filter that adapts both in center frequency and bandwidth, and/or dynamicallty changes filter coefficients.

Thus, while there have shown and described and pointed out fundamental novel features of the invention as applied to a preferred embodiment thereof, it will be understood that various omissions and substitutions and changes in the form and details of the devices illustrated, and in their operation, may be made by those skilled in the art without departing from the spirit of the invention. For example, it is expressly intended that all combinations of those elements and/or method steps which perform substantially the same function in substantially the same way to achieve the same results are within the scope of the invention. Moreover, it should be recognized that structures and/or elements and/or method steps shown and/or described in connection with any disclosed form or embodiment of the invention may be incorporated in any other disclosed or described or suggested form or embodiment as a general matter of design choice. It is the intention, therefore, to be limited only as indicated by the scope of the claims appended hereto.

1. A method for adaptively filtering a clutter signal from a received echo signal from which spectral Doppler data will be generated, comprising the steps of:
transmitting ultrasonic waves into a sample volume;
receiving an echo signal from the sample volume;
making a short-term averaged correlation estimate of a clutter signal in the received echo signal;
adaptively filtering out the clutter signal from the received echo signal by using the short-term averaged correlation estimate and a clutter filter, wherein filter coefficients of the clutter filter are not changed by said step of adaptively filtering; and
analyzing the filtered signal in order to generate spectral Doppler data.

2. The method of claim 1, wherein the clutter filter is an IIR filter.

3. The method of claim 1, wherein a stopband of the clutter filter is fixed in at least one of width and center frequency.

4. The method of claim 1, wherein the step of adaptively filtering out the clutter signal from the received echo signal comprises the step of:
adapting the filtering to an estimated clutter signal at least 4 times a second.

5. The method of claim 1, wherein the step of making a short-term averaged correlation estimate comprises the step of:
low-pass filtering the received echo signal and using the low-pass filtered echo signal to make the short-term averaged correlation estimates.

6. The method of claim 1, wherein the step of making a short-term averaged correlation estimate comprises the steps of:
forming instantaneous correlation estimates of the clutter signal in the received echo signal; and
short-term averaging the instantaneous correlation estimates to make the short-term averaged correlation estimate.

7. The method of claim 6, wherein the step of forming instantaneous correlation estimates comprises the step of:
forming an instantaneous correlation estimate by multiplying a sample of the clutter signal by a complex conjugate of a previous sample.

8. The method of claim 7, wherein the previous sample is immediately previous to the current sample (instantaneous lag 1 correlation).

9. The method of claim 7, wherein the previous sample is more than one previous to the current sample.

10. The method of claim 6, wherein the step of short-term averaging the instantaneous correlation estimates to make the short-term averaged correlation estimate is performed by either a moving average filter (FIR) or an autoregressive (IIR) filter.

11. The method of claim 6, wherein the step of short-term averaging the instantaneous correlation estimates to make the short-term averaged correlation estimate is performed less than every sample, and there is enough overlap in the averaging to ensure that successive estimates change gradually.

12. The method of claim 6, wherein the step of short-term averaging the instantaneous correlation estimates to make the short-term averaged correlation estimate is performed such that the short-term averaged correlation estimate is limited to low frequency (small angle) to avoid adaptively filtering out rapid motion.

13. The method of claim 1, wherein the step of adaptively filtering out the clutter signal from the received echo signal comprises the step of at least one of:
effectively changing a center frequency of a stopband of the clutter filter; and
effectively changing a width of the stopband of the clutter filter.

14. The method of claim 13, wherein the clutter filter has a fixed stopband, and wherein the step of effectively changing a center frequency of a stopband of the clutter filter comprises the steps of:
complex rotating the received echo signal so that the clutter signal, as estimated by the short-term averaged correlation estimate, is shifted to the fixed stopband of the clutter filter;
filtering the complex rotated signal with the clutter filter;
complex rotating the clutter filtered signal so that the output signal is shifted back to the original location of the received echo signal.

15. The method of claim 14, wherein a complex rotation factor used in the step of complex rotating the clutter filtered signal so that the output signal is shifted back to the original location of the received echo signal is a variable-frequency local oscillator (LO), which is a unit-magnitude phasor whose phase is updated every sample by multiplying itself by the unit-magnitude version of a current short-term averaged correlation estimate.

16. The method of claim 14, wherein a complex rotation factor used in the step of complex rotating the received echo signal so that the clutter signal, as estimated by the short-term averaged correlation estimate, is shifted to the fixed stopband of the clutter filter is a complex conjugate of a complex rotation factor used in the step of complex rotating the clutter filtered signal so that the output signal is shifted back to the original location of the received echo signal.

17. The method of claim 13, wherein the clutter filter comprises a plurality of clutter filters, and wherein the step of effectively changing a width of the stopband of the clutter filter comprises the step of:
inputting the received echo signal into the plurality of clutter filters, wherein each of the plural clutter filters has a different fixed stopband.

18. The method of claim 17, wherein the step of effectively changing a width of the stopband of the clutter filter comprises the step of:
selecting an output from one of the plurality of clutter filters, wherein said selection is based upon a current short-term averaged correlation estimate.

19. The method of claim 17, wherein the step of effectively changing a width of the stopband of the clutter filter comprises the step of:
interpolating an output from plural outputs from the plural clutter filters, wherein said interpolation is based upon a current short-term averaged correlation estimate.

20. As system for adaptively filtering a clutter signal from a received echo signal from which spectral Doppler data will be generated, comprising:
an estimator for making a short-term averaged correlation estimate of a clutter signal in an echo signal received from a sample volume into which ultrasonic waves had been transmitted; and
a means for adaptively filtering out the clutter signal from the received echo signal by using the short-term averaged correlation estimate and a clutter filter with fixed filter coefficients.
